(19) United States
(12) Patent Application Publication
(10) Pub. No.: US 2004/0086874 A1
(43) Pub. Date: May 6, 2004

(54) Devices and methods for holding a biopolymeric array

(76) Inventor: Russell A. Parker, San Jose, CA (US)

Correspondence Address: Agilent Technologies, Inc., Legal Department, DL429, Intellectual Property Administration, P.O. Box 7599, Loveland, CO, 80537-0599, US

(21) Appl. No.: 10/286,649

(22) Filed: Oct. 31, 2002

(51) Int. Cl. C12Q 1/68; G01N 33/53; G01N 33/542; B23Q 3/00; C12M 1/34

(52) U.S. Cl. 435/6; 435/7.9; 435/287.2; 269/287

(57) ABSTRACT

Devices and methods for holding at least one array are provided. The subject devices are characterized by having a housing having at least one array therein and an absorbing material associated with the housing that is capable of absorbing molecules within the housing deleterious to the array(s) held therein. The seal may be resealable and/or may be a hermetic seal. The subject methods include packaging at least one array in a subject array holding device. Also provided are methods for using an array that is held in a subject array holding device in an array assay. Kits for use in the subject methods are also provided.

### FIELD OF THE INVENTION

The field of this invention is biopolymeric arrays.

### BACKGROUND OF THE INVENTION

Arrays of biopolymeric molecules, e.g., “gene chips,” have become an increasingly important tool in the biotechnology industry and related fields. These biopolymeric arrays, in which a plurality of distinct or different biopolymers are positioned on a solid support surface in the form of an array or pattern, find use in a variety of different fields, e.g., genomics (in sequencing by hybridization, SNP detection, differential gene expression analysis, identification of novel genes, gene mapping, finger printing, etc.) and proteomics. Each distinct polymeric sequence of the array is typically present as a composition of multiple copies of the polymer on a substrate surface, e.g., as a spot or feature, on the surface of a substrate. The surface-bound biopolymers or probes may be oligonucleotides, peptides, polypeptides, proteins, antibodies or other molecules capable of binding with target biomolecules in the solution.

These arrays are used in array assays wherein a solution suspected of containing an analyte or target molecule(s) (“targets”) that bind with the attached probes is placed in contact with the bound probes under conditions sufficient to promote binding of targets in the solution to the complementary probes on the substrate to form a binding complex that is bound to the surface of the substrate. The pattern of binding by target molecules to probe features or spots on the substrate produces a pattern which is detected. This detection of binding complexes provides desired information about the target biomolecules in the solution.

The binding complexes may be detected by reading or scanning the array with, for example, optical means, although other methods may also be used, as appropriate for the particular assay. For example, laser light may be used to excite fluorescent labels attached to the targets, generating a signal only in those spots on the array that have a labeled target molecule bound to a probe molecule. This pattern may then be digitally scanned for computer analysis. Such patterns can be used to generate data for biological assays such as the identification of drug targets, single-nucleotide polymorphism mapping, monitoring samples from patients to track their response to treatment, assessing the efficacy of new treatments, etc.

However, the biopolymers of an array can be sensitive to airborne contaminants such as water, inorganic chloride, e.g., chlorine, chlorine dioxide, hydrogen chloride, hydrogen fluoride, active sulfur compounds, e.g., hydrogen sulfide, elemental sulfur, organic sulfur compounds, sulfur oxides, nitrogen oxides, ammonia, amines, ammonium ions, ozone, free radicals, organic compounds, e.g., acetic acid, halocarbons, MTBE, carbon dioxide, etc. In other words, molecules in the atmosphere that come in contact with the arrays may have an adverse affect on the biopolymeric features of the array. Since there are relatively few biopolymer molecules in a spot or feature because the typical process employed for producing such spots or features produces small, nearly monolayers of molecules, very little contaminant may negatively affect the array. In many instances, the biopolymers of the array are affected to such an extent that the results of the array assay are compromised, e.g., the detected signal may be decreased giving erroneous assay results. Array exposure to deleterious contaminants occurs at a number of different phases in an array's life cycle. For example, an array may be exposed to these deleterious molecules during the manufacture thereof, e.g., between manufacturing steps, during manufacturing down times such as shift changes, etc. Likewise, an array may also be exposed to deleterious molecules after manufacture, i.e., during transport to a customer site and also once in the hands of a user, e.g., before an array assay is performed, as well as after an array assay has been performed for example while the array is waiting to be read by an array scanner. Similar degradation problems exist for dyes that are used in detectable labels associated with target molecules, as described above.

To address the problem of harmful atmospheric exposure, a number of solutions exits, however each attempted solution suffers from significant disadvantages. One such solution involves storing the arrays at relatively low temperatures, i.e., temperatures below room temperature such as 4° C., −20° C. or −80° C. However, it is costly and impractical to provide this environment for many arrays at one time as refrigeration units for this purpose can be expensive and a significant number of refrigeration units would be needed to store the arrays. Furthermore, the refrigeration units would take up significant and oftentimes expensive laboratory or near-laboratory space. Additionally, arrays stored at such low temperatures must be allowed to equilibrate to room temperature before use, otherwise condensation will occur thereon which may react adversely with the array's chemistry. Furthermore, a refrigerated cold environment is not portable and may not be positioned near a scanner, so a protocol to scan the arrays after they have equilibrated to room temperature, but before they have been in the laboratory environment too long, is often difficult to follow.

Another solution that has been proposed involves storing the arrays in an inert atmosphere. However, this solution is not practical to implement in most manufacturing facilities, transport carriers and laboratories, as special storage cabinets having continuous flow nitrogen must be used. These special set-ups are expensive, especially for the size of a set-up that would be needed for a manufacturing facility, transport carrier or laboratory. Furthermore, the size of such a set-up is also limited as entry-type cabinets could pose a risk of asphyxiation.

Storing arrays for relatively short periods of time is another solution that has been proposed. However, this proposal becomes impractical for normal operations, as typically there are delays in the processing, handling and analysis of arrays. To overcome these delays, duplicates of costly equipment for processing and analysis may be required to be purchased, the expense and space requirements of which would likely be prohibitive for most operations. However, even the purchase of duplicate equipment will not solve the problem entirely as certain arrays, e.g., quality control arrays, scanner calibration arrays and arrays maintained as historical records may require longer periods of storage. Furthermore, precisely pin pointing the maximum amount of time an array can be exposed to the atmosphere before being adversely affected by atmospheric elements may be difficult, time consuming, labor intensive and will vary depending on the particular array chemistry.

Attempts have also been proposed to combat the problem as it relates to the degradation of the dyes used in the array assays. One such proposed solution involves processing the arrays with dyes that are more stable than those currently used and thus not prone to the above-described degradation. However, to date such a dye has not been found that is universally successful. Furthermore, it is costly to implement the necessary changes to procedures, e.g., in manufacturing, array assay reagents, scanners, etc., to incorporate new chemistries. Also, there is risk that data obtained from prior arrays will not precisely correlate with data obtained from arrays with new chemistries, thus necessitating some or many experiments to be repeated. Furthermore, a user will most likely be hesitant to switch to a new, unknown system.

Accordingly, there continues to be an interest in the development of new devices and methods to hold and store arrays such that the arrays are not in constant contact with molecules in the atmosphere that are harmful to the arrays. Of particular interest is the development of such devices and methods that are cost effective, easy to use, effective at providing a controlled atmosphere for the arrays, and which may be employed in various phases of an array's lifetime.

### SUMMARY OF THE INVENTION

Devices and methods for holding at least one array are provided. The subject devices are characterized by having a housing having at least one array therein and an absorbing material associated with the housing that is capable of absorbing molecules within the housing deleterious to the array(s) held therein. The seal may be resealable and/or may be a hermetic seal. The subject methods include packaging at least one array in a subject array holding device. Also provided are methods for using an array that is held in a subject array holding device in an array assay. Kits for use in the subject methods are also provided.

### BRIEF DESCRIPTIONS OF THE DRAWINGS

FIG. 1 shows an exemplary embodiment of a subject array holder having a rectangular shape.

FIG. 2 shows another exemplary embodiment of a subject array holder having a square shape.

FIG. 3 shows an exemplary embodiment of a subject array holder having a disc shape.

FIG. 4 shows an exemplary embodiment of a subject array holder wherein the bottom and the cover are not completely separable.

FIG. 5 shows another exemplary embodiment of a subject array holder wherein the bottom and the cover are not completely separable.

FIG. 5A shows a cross-sectional view of the array holder of FIG. 5 having an array positioned atop bumpers located on a first surface.

FIG. 5B shows a cross-sectional view of the array holder of FIG. 5 having an array positioned between bumpers located on a first surface and a second surface.

FIG. 6 shows an exemplary embodiment of a subject array holder wherein the bottom and the cover are completely separable.

FIG. 7 shows a cross-sectional view of an exemplary subject array holder having spacers to separate array substrates from each other.

FIG. 8 shows a cross-sectional view of an exemplary subject array holder having spacers positioned as ledges or rails or shelves inside the array holder.

FIG. 9 shows an exemplary embodiments of a subject device having absorbing material configured as insertable strips.

FIG. 10 shows an exemplary embodiment of a subject device having absorbing material configured as an insertable sheet or layer.

FIG. 11A shows an exemplary embodiment of an insertable sleeve being inserted into a subject device.

FIG. 11B shows the insertable sleeve of FIG. 11A fully inserted into the device to surround an array in the device.

FIG. 12 shows an exemplary embodiment of a subject device wherein the absorbing material is integral to the device.

FIG. 13 shows an exemplary embodiment of a subject device wherein the absorbing material forms the housing.

FIG. 14 shows a graph of an array that was not packaged according to the subject invention, over about three months (control).

FIGS. 15A and 15B show graphs of arrays that were packaged according to the subject invention, over about three months.

FIGS. 16A and 16B show graphs of yet more arrays that were packaged according to the subject invention, over about three months.

### DEFINITIONS

The term “nucleic acid” as used herein means a polymer composed of nucleotides, e.g. deoxyribonucleotides or ribonucleotides, or compounds produced synthetically (e.g. PNA as described in U.S. Pat. No. 5,948,902 and the references cited therein) which can hybridize with naturally occurring nucleic acids in a sequence specific manner analogous to that of two naturally occurring nucleic acids, e.g., can participate in hybridization reactions, i.e., cooperative interactions through Pi electrons stacking and hydrogen bonds, such as Watson-Crick base pairing interactions, Wobble interactions, etc.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 100 nucleotides and up to 200 nucleotides in length.

The term “polynucleotide” as used herein refers to single or double stranded polymer composed of nucleotide monomers of generally greater than 100 nucleotides in length.

The term “monomer” as used herein refers to a chemical entity that can be covalently linked to one or more other such entities to form an oligomer. Examples of “monomers” include nucleotides, amino acids, saccharides, peptides, and the like. In general, the monomers used in conjunction with the present invention have first and second sites (e.g., C-termini and N-termini, or 5′ and 3′ sites) suitable for binding to other like monomers by means of standard chemical reactions (e.g., condensation, nucleophilic displacement of a leaving group, or the like), and a diverse element which distinguishes a particular monomer from a different monomer of the same type (e.g., an amino acid side chain, a nucleotide base, etc.). The initial substrate-bound monomer is generally used as a building-block in a multi-step synthesis procedure to form a complete ligand, such as in the synthesis of oligonucleotides, oligopeptides, and the like.

The term “oligomer” is used herein to indicate a chemical entity that contains a plurality of monomers. As used herein, the terms “oligomer” and “polymer” are used interchangeably. Examples of oligomers and polymers include polydeoxyribonucleotides (DNA), polyribonucleotides (RNA), other polynucleotides which are C-glycosides of a purine or pyrimidine base, polypeptides (proteins), polysaccharides (starches, or polysugars), and other chemical entities that contain repeating units of like chemical structure.

The terms “nucleoside” and “nucleotide” are intended to include those moieties which contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present, and, thus, the description includes structures wherein a non-hydrogen substituent is present and structures wherein a non-hydrogen substituent is not present.

An “array,” includes any two-dimensional or substantially two-dimensional (as well as a three-dimensional) arrangement of addressable regions bearing a particular chemical moiety or moieties (e.g., biopolymers such as polynucleotide or oligonucleotide sequences (nucleic acids), polypeptides (e.g., proteins), carbohydrates, lipids, etc.) associated with that region. In the broadest sense, the preferred arrays are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus). Sometimes, the arrays are arrays of polypeptides, e.g., proteins or fragments thereof.

Any given substrate may carry one, two, four or more or more arrays disposed on a front surface of the substrate. Depending upon the use, any or all of the arrays may be the same or different from one another and each may contain multiple spots or features. A typical array may contain more than ten, more than one hundred, more than one thousand more ten thousand features, or even more than one hundred thousand features, in an area of less than 20 cm² or even less than 10 cm². For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Each array may cover an area of less than 100 cm², or even less than 50 cm², 10 cm² or 1 cm². In many embodiments, the substrate carrying the one or more arrays will be shaped generally as a rectangular solid (although other shapes are possible), having a length of more than about 4 mm and less than about 1 m, usually more than about 4 mm and less than about 600 mm, more usually less than about 400 mm; a width of more than about 4 mm and less than about 1 m, usually less than about 500 mm and more usually less than about 400 mm; and a thickness of more than about 0.01 mm and less than about 5.0 mm, usually more than about 0.1 mm and less than about 2 mm and more usually more than about 0.6 mm and less than about 1.5 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, a substrate may transmit at least 20%, or 50% (or even at least 70%, 90%, or 95%), of the illuminating light incident on the front as may be measured across the entire integrated spectrum of such illuminating light or alternatively at 532 nm or 633 nm.

An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probe” may be the one which is to be evaluated by the other (thus, either one could be an unknown mixture of polynucleotides to be evaluated by binding with the other). A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there exists intervening areas which lack features of interest. An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

“Remote location,” means a location other than the location at which the array is present and hybridization occurs. For example, a remote location could be another location (e.g., office, lab, etc.) in the same city, another location in a different city, another location in a different state, another location in a different country, etc. As such, when one item is indicated as being “remote” from another, what is meant is that the two items are at least in different rooms or different buildings, and may be at least one mile, ten miles, or at least one hundred miles apart.

“Communicating” information references transmitting the data representing that information as electrical signals over a suitable communication channel (e.g., a private or public network).

“Forwarding” an item refers to any means of getting that item from one location to the next, whether by physically transporting that item or otherwise (where that is possible) and includes, at least in the case of data, physically transporting a medium carrying the data or communicating the data.

“Transporting” in an item refers to any means of getting the item from one site to the next, i.e., physically moving or shipping the item to a second.

### DETAILED DESCRIPTION OF THE INVENTION

Devices and methods for holding at least one array are provided. The subject devices are characterized by having a housing having at least one array therein and an absorbing material associated with the housing that is capable of absorbing molecules within the housing deleterious to the array(s) held therein. The seal may be resealable and/or may be a hermetic seal. The subject methods include packaging at least one array in a subject array holding device. Also provided are methods for using an array that is held in a subject array holding device in an array assay. Kits for use in the subject methods are also provided.

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

As summarized above, the subject invention provides devices and methods of holding at least one array so as to minimized damage to the held array(s) from reactive agents the atmosphere harmful to arrays. In further describing the subject invention, a review of representative arrays that may be used with the subject invention will be described first to provide a proper foundation for describing the invention. Next, the subject devices and methods will be described in detail, followed by a description of kits for use with the subject methods.

### REPRESENTATIVE BIOPOLYMERIC ARRAYS

As mentioned above, the devices and methods of the subject invention are directed to holding at least one array in an environment that protects the array(s) from harmful elements in the atmosphere. Such arrays find use in a variety of applications, including gene expression analysis, drug screening, nucleic acid sequencing, mutation analysis, and the like. These arrays include a plurality of ligands or molecules or probes (i.e., binding agents or members of a binding pair) deposited onto the surface of a substrate in the form of an “array” or pattern.

The subject arrays include at least two distinct polymers that differ by monomeric sequence attached to different and known locations on the substrate surface. Each distinct polymeric sequence of the array is typically present as a composition of multiple copies of the polymer on a substrate surface, e.g., as a spot or feature on the surface of the substrate. The number of distinct polymeric sequences, and hence spots or similar structures, present on the array may vary, where a typical array may contain more than about ten, more than about one hundred, more than about one thousand, more than about ten thousand or even more than about one hundred thousand features in an area of less than about 20 cm² or even less than about 10 cm². For example, features may have widths (that is, diameter, for a round spot) in the range from about 10 μm to about 1.0 cm. In other embodiments, each feature may have a width in the range from about 1.0 μm to about 1.0 mm, usually from about 5.0 μm to about 500 μm and more usually from about 10 μm to about 200 μm. Non-round features may have area ranges equivalent to that of circular features with the foregoing width (diameter) ranges. At least some, or all, of the features are of different compositions (for example, when any repeats of each feature composition are excluded, the remaining features may account for at least about 5%, 10% or 20% of the total number of features). Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents, but may not be present when, for example, photolithographic array fabrication process are used. It will be appreciated though, that the interfeature areas, when present, could be of various sizes and configurations. The spots or features of distinct polymers present on the array surface are generally present as a pattern, where the pattern may be in the form of organized rows and columns of spots, e.g. a grid of spots, across the substrate surface, a series of curvilinear rows across the substrate surface, e.g. a series of concentric circles or semi-circles of spots, and the like.

In the broadest sense, the arrays are arrays of polymeric or biopolymeric ligands or molecules, i.e., binding agents, where the polymeric binding agents may be any of: peptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like.

The arrays may be produced using any convenient protocol. Various methods for forming arrays from pre-formed probes, or methods for generating the array using synthesis techniques to produce the probes in situ, are generally known in the art. See, for example, Southern, U.S. Pat. No. 5,700,637; Pirrung, et al., U.S. Pat. No. 5,143,854 and Fodor, et al. (1991) Science 251:767-777, the disclosures of which are incorporated herein by reference and PCT International Publication No. WO 92/10092. For example, probes can either be synthesized directly on the solid support or substrate to be used in the array assay or attached to the substrate after they are made. Arrays may be fabricated using drop deposition from pulse jets of either polynucleotide precursor units (such as monomers) in the case of in situ fabrication, or the previously obtained polynucleotide. Such methods are described in detail in, for example, the previously cited references including U.S. Pat. Nos. 6,242,266, 6,232,072, 6,180,351, 6,171,797, and 6,323,043; and U.S. patent application Ser. No. 09/302,898 filed Apr. 30, 1999 by Caren et al., and the references cited therein, the disclosures of which are herein incorporated by reference. Other drop deposition methods may be used for fabrication. Also, instead of drop deposition methods, photolithographic array fabrication methods may be used such as described in U.S. Pat. Nos. 5,599,695, 5,753,788, and 6,329,143, the disclosures of which are herein incorporated by reference. As mentioned above, interfeature areas need not be present, particularly when the arrays are made by photolithographic methods as described in those patents.

Immobilization of the probe to a suitable substrate may be performed using conventional techniques. See, e.g., Letsinger et al. (1975) Nucl. Acids Res. 2:773-786; Pease, A. C. et al., Proc. Nat. Acad. Sci. U.S.A., 1994, 91:5022-5026, and Oligonucleotide Synthesis, a Practical Approach,” Gait, M. J. (ed.), Oxford, England: IRL Press (1984). The surface of a substrate may be treated with an organosilane coupling agent to functionalize the surface. See, e.g., Arkins, ASilane Coupling Agent Chemistry,” Petrarch Systems Register and Review, Eds. Anderson et al. (1987) and U.S. Pat. No. 6,258,454.

A variety of solid supports or substrates may be used, upon which an array may be positioned. Any given substrate may carry one, two, four or more or more arrays disposed on a front surface of the substrate. Depending upon the use, any or all of the arrays may be the same or different from one another and each may contain multiple spots or features. For example, a plurality of arrays may be stably associated with one substrate, where the arrays are spatially separated from some or all of the other arrays associated with the substrate.

The array substrate may be selected from a wide variety of materials including, but not limited to, natural polymeric materials, particularly cellulosic materials and materials derived from cellulose, such as fiber containing papers, e.g., filter paper, chromatographic paper, etc., synthetic or modified naturally occurring polymers, such as nitrocellulose, cellulose acetate, poly (vinyl chloride), polyamides, polyacrylamide, polyacrylate, polymethacrylate, polyesters, polyolefins, polyethylene, polytetrafluoro-ethylene, polypropylene, poly (4-methylbutene), polystyrene, poly(ethylene terephthalate), nylon, poly(vinyl butyrate), cross linked dextran, agarose, etc.; either used by themselves or in conjunction with other materials; fused silica (e.g., glass), bioglass, silicon chips, ceramics, metals, and the like. For example, substrates may include polystyrene, to which short oligophosphodiesters, e.g., oligonucleotides ranging from about 5 to about 50 nucleotides in length, may readily be covalently attached (Letsinger et al. (1975) Nucl. Acids Res. 2:773-786), as well as polyacrylamide (Gait et al. (1982) Nucl. Acids Res. 10:6243-6254), silica (Caruthers et al. (1980) Tetrahedron Letters 21:719-722), and controlled-pore glass (Sproat et al. (1983) Tetrahedron Letters 24:5771-5774). Additionally, the substrate can be hydrophilic or capable of being rendered hydrophilic.

Suitable array substrates may exist, for example, as sheets, tubing, spheres, containers, pads, slices, films, plates, slides, strips, disks, etc. The substrate is usually flat, but may take on alternative surface configurations. The substrate can be a flat glass substrate, such as a conventional microscope glass slide, a cover slip and the like. Common substrates used for the arrays of probes are surface-derivatized glass or silica, or polymer membrane surfaces, as described in Maskos, U. et al., Nucleic Acids Res, 1992, 20:1679-84 and Southern, E. M. et al., Nucleic acids Res, 1994, 22:1368-73.

Each array may cover an area of less than about 100 cm², or even less than about 50 cm², 10 cm² or 1 cm². In many embodiments, the substrate carrying the one or more arrays will be shaped generally as a rectangular solid (although other shapes are possible), having a length of more than about 4 mm and less than about 1 m, usually more than about 4 mm and less than about 600 mm, more usually less than about 400 mm; a width of more than about 4 mm and less than about 1 m, usually less than about 500 mm and more usually less than about 400 mm; and a thickness of more than about 0.01 mm and less than about 5.0 mm, usually more than about 0.1 mm and less than about 2 mm and more usually more than about 0.6 and less than about 1.5 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least about 20%, or about 50% (or even at least about 70%, 90%, or 95%), of the illuminating light incident on the substrate as may be measured across the entire integrated spectrum of such illuminating light or alternatively at 532 nm or 633 nm.

### Devices for Holding at Least One Bipolymeric Array

As summarized above, the subject invention includes array holding devices for holding at least one array therein, such as one or more arrays of the type described above, i.e., biopolymeric arrays. The array holders of the subject invention provide protection to the held arrays(s) from harmful atmospheric elements. By “harmful” or “deleterious” (herein used interchangeably) it is generally meant that such molecule(s) cause a decrease in signal intensity of a dye molecule and/or inhibits the binding efficiency or capability of a binding pair to bind to each other, i.e., decreases the ability of an array probe molecule and its target complement molecule to bind to one another, by interfering with the probe molecule's ability to bind its complement. The decrease in signal intensity of a dye molecule may vary depending on a variety of factors such as the particular dye employed, the particular atmospheric element(s) acting on the dye molecule, the particular binding pair employed, the amount of time of exposure of the dye molecule to the particular atmospheric element(s), etc., where typically this decrease in signal intensity of a dye molecule ranges from about 1% to about 90% as compared to signal intensity obtained from dye molecules not exposed to such atmospheric element(s), as determined, for example, by the assay described in the Experimental Section below. Likewise, the decrease in binding efficiency of a probe molecule to bind to its complement may vary depending on a variety of factors such as the particular atmospheric element(s) acting on the probe molecule, the particular binding pair employed, the amount of time of exposure of the probe molecule to the particular atmospheric element(s), etc., where typically this decrease in binding efficiency ranges from about 1% to about 90% as compared to the binding efficiency of a binding pair not exposed to such atmospheric element(s), as determined, for example, by the assay described in the Experimental Section below.

The holder may protect the oligonucleotide probes of the arrays themselves and/or the dye molecule label, e.g., bound to the probes following a binding assay. In general, the array holders include a housing configured to hold one or more arrays inside thereof, where in many embodiments the seal is a hermetic seal. In certain embodiments, the seal may be resealable. In accordance with the subject invention, the housings includes an absorbing material, e.g., a dessicating material, which will be described in greater detail below, that absorbs molecules from the atmosphere so as to provide a controlled environment inside the housing for the arrays.

Accordingly, the array holders are configured to hold at least one array therein. As described above, arrays range in size and shape depending on the particular array. As such, the shape of the subject holders is not particularly important to the subject invention so long as the holders are capable of holding at least one array therein without damaging the array and are able to be associated with an absorbing agent. In many embodiments, the holders are configured to be easily transportable from a first location to a second location. For example, an array may be held in a particular holder from the beginning of the manufacturing process through the useful life of the array, e.g., through and after the array assay procedure up to the time the array is positioned on an array scanner to be read and in certain embodiments after an array has been read.

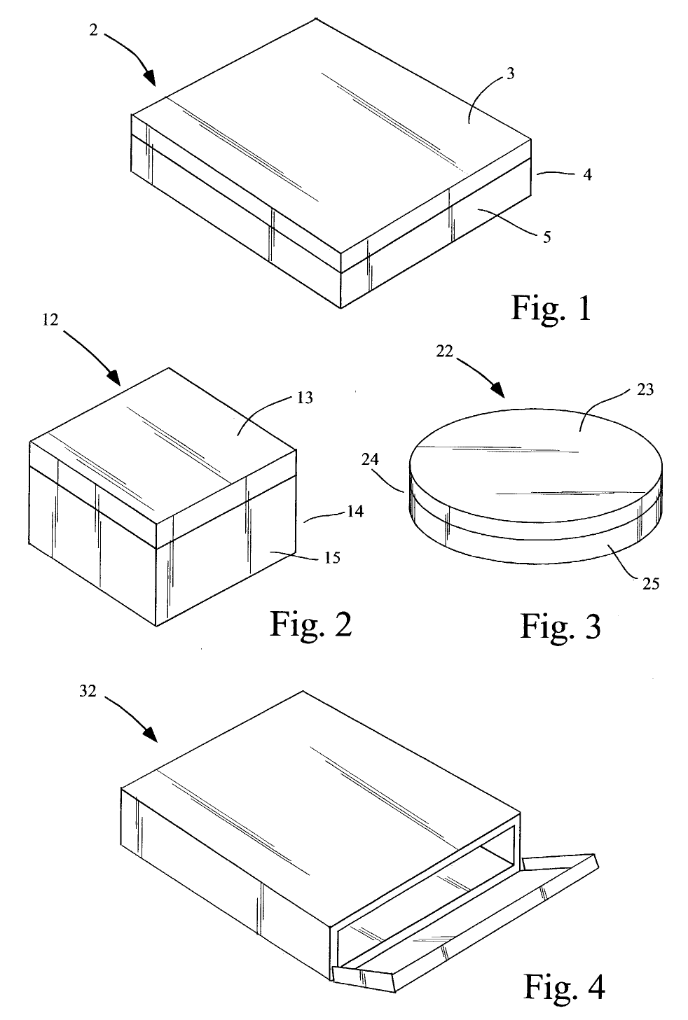

As such, the array holders of the subject invention may assume a variety of shapes ranging from simple to complex, with the only limitation being that they are suitably shaped to retain or hold at least one array therein. FIGS. 1-3 show three exemplary embodiments of the subject holders, although other shapes are possible as well, such as irregular or complex shapes. FIG. 1 shows an array holder device 2 that includes a housing 4 having a rectangular shape, FIG. 2, shows an array holder device 12 that includes a housing 14 having a square shape and FIG. 3 shows an array holder device 22 that includes a housing 24 having a circular or disc shape.

Likewise, the size of the array holders may vary depending on a variety of factors, including, but not limited to, the size of an array substrate, the number of array substrates that can be held therein, and the like. The subject array holders are typically dimensioned to hold from about 1 array substrate to about 100 array substrates or more, usually from about 5 array substrates to about 50 array substrates and more usually from about 10 array substrates to about 25 array substrates, where each array substrates may carry one or more arrays thereon as described below. Generally, the subject array holders will be sized to be easily transportable or moveable as described above. In certain embodiments of the subject devices having a substantially rectangular shape and configured to hold a single array substrate having dimensions of about 25 mm by about 75 mm by about 1 mm, the length of the array holder typically ranges from about 77 mm to about 100 mm, usually from about 80 mm to about 90 mm and more usually from about 80 mm to about 85 mm, the width typically ranges from about 27 mm to about 50 mm, usually from about 29 mm to about 40 mm and more usually from about 30 mm to about 35 mm and the thickness typically ranges from about 2 mm to about 10 mm, usually from about 3 mm to about 7 mm and more usually from about 4 mm to about 6 mm. However, these dimensions are exemplary only and may vary as required.

The subject devices may be fabricated from a wide variety of materials with the limitation that the material(s) employed do not degrade or otherwise adversely affect an array substrate or chemistry. The devices may be flexible or rigid or may be both flexible and rigid such that a portion of the device may be rigid and a portion may be flexible. Of interest are materials that are substantially impermeable to gasses and in many embodiments substantially impermeable to light such that when in a sealed configuration, light from the exterior of the device is not able of penetrate through the device to the interior thereof. Examples of materials which may be used to fabricate the devices include, but are not limited to, plastics such as polytetrafluoroethylene, polypropylene, polystyrene, polycarbonate, PVC, and blends thereof, stainless steel and alloys thereof, siliceous materials, e.g., glasses, fused silica, ceramics and the like, and other metals such as aluminum and its alloys. The subject devices or any component thereof may be manufactured to be re-useable or single use.

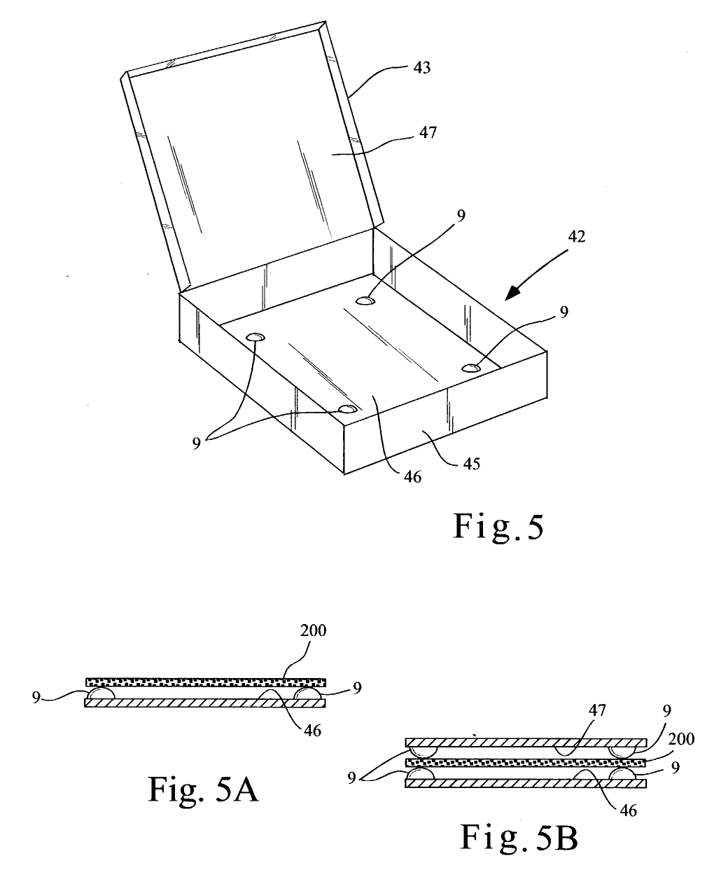

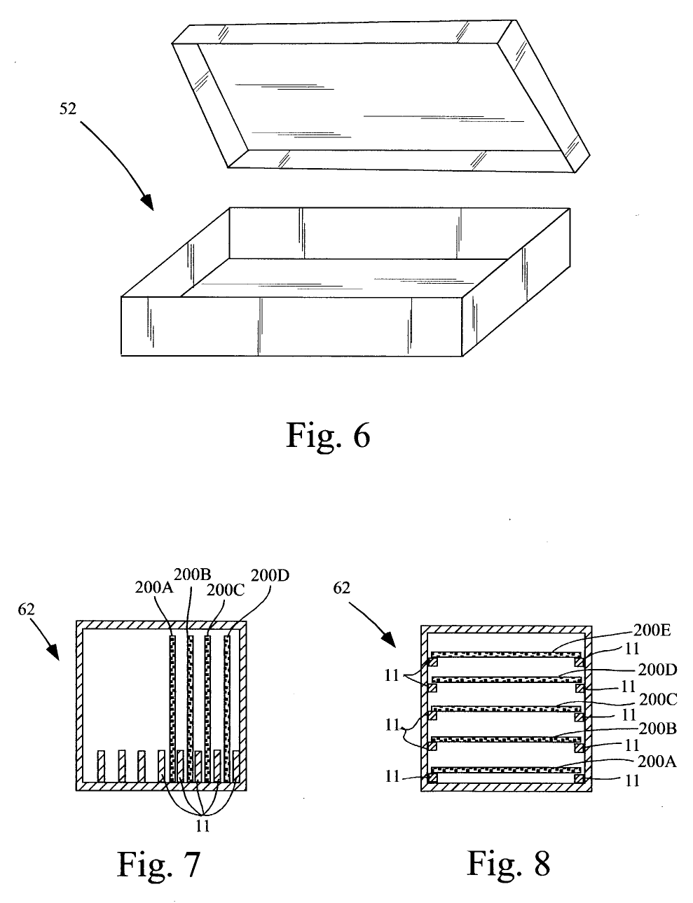

As summarized above, the subject array holders may be resealable such that they may be opened, one or more arrays may be removed or added, and then resealed, one or more times. As shown in FIGS. 1-3, the array holders include at least two mateable components: a bottom component 5, 15 and 25, respectively, and a mateable cover or lid 3, 13 and 23, respectively, configured such that the two components form an enclosure when sealed together in a closed configuration. The two components may be integrated together such that they may be joined by a hinge or may be molded or extruded from a single piece of material or the like or may be completely separable. FIGS. 4 and 5 show exemplary embodiments of array holders 32 and 42, respectively, having bottoms and covers constructed as integrated units, i.e., not completely separable. FIG. 6 shows an exemplary embodiment of an array holder 52 having a bottom and cover that are completely separable.

Regardless of whether the bottom and cover are integrated or not completely separable or whether they are completely separable, in many embodiments the devices of the subject invention provide a hermetic seal when the bottom and the cover are sealed together to form a closed housing. The hermetic seal may be accomplished in a variety of manners, where the choice of such may be dictated by a variety of factors such as the shape of the housing, the size of the housing and the materials of the housing. Manners of providing hermetic seals are well known in the art and will not be repeated here. For example, a gasket, o-ring, multiple material interfaces, threaded arrangement, viscous sealant, or the like, may be used to effect a hermetic seal. In those embodiments having resealable housings, a hermetic seal is typically provided each time a device is re-sealed.

The housings of the subject devices may further include one or more optional features or structures to facilitate holding at least one array therein without damaging the array. For example, FIG. 5 shows optional bumpers or protrusions 9 positioned on surface 46 of bottom 45, upon which an array may be placed so that the array is maintained a fixed distance from the surface. This is better illustrated in the cross sectional view of FIG. 5A which illustrates array 200 positioned on bumpers 9 of bottom 45. Bumpers may additionally or instead be positioned on surface 47 of cover 43, as best illustrated in cross section in FIG. 5B.

As mentioned above, more than one array substrate, each carrying one or more arrays thereon, may be positioned in a subject array holder at the same time. As such, it may be desirable to provide a space between the array substrates so that they do not contact and damage each other. In certain embodiments, optional separators may be positioned between adjacent array substrates in an array holder to provide a physical barrier between the arrays. FIG. 7 shows a cross section of an array holder 62 having spacers 11 positioned therein to separate the arrays 200a, 200b, 200c and 200d, from each other. The spacers may be positioned as ledges or rails or shelves inside an array holder, as shown in cross section in FIG. 8, upon which arrays 200a, 200b, 200c, 200d and 200e are positioned.

A feature of the subject devices is that they include a material that protects the array(s) held therein from deleterious reactive species in the environment to which they would otherwise be exposed. In other words, the subject array holders include a reactive material that removes, e.g., by chemical or physical reaction, molecules inside the holder that would otherwise chemically react or physically alter an array, negatively impacting further processing and/or data analysis. Deleterious vapors and gases and the like that may be absorbed by the subject absorbing materials include, but are not limited to: water, inorganic chloride, e.g., chlorine, chlorine dioxide, hydrogen chloride, hydrogen fluoride, active sulfur compounds, e.g., hydrogen sulfide, elemental sulfur, organic sulfur compounds, sulfur oxides, nitrogen oxides, ammonia, amines, ammonium ions, ozone, free radicals, organic compounds, e.g., acetic acid, halocarbons, MTBE, carbon dioxide and other undesirable vapors and gases.

The subject materials may also, in certain embodiments, emit molecules that may be advantageous to an array such as, but not limited to: antioxidants, such as butylated hydroxytoluene, dimethylamine, and phenolic aldehydes; anti-reducing agents, such as chlorine, benzoyl peroxide, organic halides and tetranitromethane; anit-acid agents such as amides; anti-base reagents such as acetic acid; and anti-radical reagents such as chlorides, hydroquinone, diphenylpicrylhydrazine, diphenyamine, and t-butylcatechol.

The above-described absorption material may be associated with the housing in a variety of ways. In certain embodiments, the absorbing material is integral with the housing such that it is embedded therein, co-molded, laminated or extruded therewith, or otherwise entrapped within the housing itself. In other words, the housing may be made partially or entirely of the absorbing material such that at least portions of the housing are formed by the subject absorbing agents and in some instances all of the housing is formed by the subject absorbing agents. In certain other embodiments, or in addition to being integral with the housing, the absorbing agent may be a separate component such as a plug ball or plug capsule or the like, strip, liner, film or layer, e.g., with an adhesive backing, laminate, coating, sheet, or insertable sleeve that is associated with positioned in the interior of the housing. For example, such completely separable absorbing materials may simply be placed in the interior of the array holder. However, in this case, the absorbing material may be prone to move about the interior of the array during movement of the array holder and thus may damage an array. Accordingly, absorbing material may be fixed to a location in the interior of an array holder using any convenient protocol such as employing adhesive or the like or the absorbing agent may be press fit into a location of the bottom and/or cover of the housing, may be laminated thereto, etc. In certain embodiments, the housing may be shrunk, e.g., using a thermal shrink fit protocol, about the absorbing material.

Of interest are absorbing materials that include a polymer and an absorbing or active agent which are combined to provide a blended absorbent material. In certain embodiments the absorbing material also includes channels open at the surface of the polymeric base. Such absorbing materials are described in detail in, for example, U.S. Pat. Nos. 5,529,686, 5,543,270, 6,022,924, 6,316,520, 6,279,736, 6,211,446, 6,214,255, 6,194,079, 6,174,952, 6,130,263, 6,124,006, 6,080,350, 5,911,937 and 6,177,183, the disclosures of which are herein incorporated by reference. Absorbing materials provided by CSP Technologies of Auburn, Ala. are of interest, e.g., CSP Technologies part nos. 1238 and 1239. As absorbing materials of interest are described in great detail in the above noted patents, such will not be herein repeated, however examples of each component of the absorbing materials will now be briefly described in turn.

### Polymer Base

As described above, in the subject absorbing materials, the absorbing agents and optional channeling agents are entrained in a polymeric base. A variety of polymeric bases may be used, where in many embodiment the polymer is a substantially water insoluble polymer. Examples of suitable polymeric materials include polyolefins such as polypropylene and polyethylene, polyisoprenes, polybutadienes, polybutenes, polysiloxanes, polycarbonates, polyamides, ethylene-vinyl acetate copolymers, ethylene-methacrylate copolymer, poly(vinyl chloride), polystyrenes, polyesters, polyanhydrides, polyacrylonitriles, polysulfones, polyacrylic esters, acrylics, polyurethanes, polyacetals, polypropylene maleic anhydrides, polyethylene maleic anhydrides, polystyrene maleic anhydrides, polyethylene acrylic acids, polyethylene-uretheres, polyethylene-EVOH and polyethylene-nylons.

Other suitable polymeric materials include grafted polyolefins, polyamides, ethylene-vinyl acetate partially hydrolyzed polymers, ethylene-methacrylate partially hydrolyzed polymers, grafted polyvinylchlorides, grafted polystyrenes, polyester amides, polyacrylic partially hydrolyzed esters, acrylonitrile-butadiene-styrenes, cellulosics, ethylene vinyl alcohols, fluoroplastics, ionomers, liquid crystal polymers, polyacrylates, polyamide-imides, polybutylenes, polyektones, polyetheretherketones, polyetherimides, polyethersulfones, polyethylenechlorinates, polyimides, polymethylpentenes, polyphenylene oxides, polyphenylene sulfides, polyphthalamides, polyvinylidene chlorides, and thermosplastic elastomers. In certain embodiments, the polymer may be an adhesive such as light curing adhesive, epoxy and/or hot melt adhesives. It will be obvious that a combination of two or more materials may be employed.

### Absorbing Agents

The absorbing or active agent(s) employed in the subject invention is chosen to achieve absorption of atmospheric elements that are deleterious to an array such as, but not limited to, harmful acids, bases, corrosives, oxidizing gasses, reducing gases, etc. By “absorption” of an atmospheric element it is meant herein generally to include not just physical absorption of an element, but also any chemical and/or physical action that removes or otherwise renders a deleterious atmospheric molecule inactive or incapable of adversely affecting array. Typically, the absorbing agent(s) is distributed substantially uniformly with respect to the polymeric base. However, the present invention also contemplates alternative configurations such that the absorbing agent(s) is not distributed substantially uniformly with respect to the polymeric base, i.e., distributed non-uniformly with respect to the polymeric base.

Elements that may be absorbed and/or otherwise rendered inactive by the subject absorbing agent(s) include, but are not limited to: water; inorganic chloride, e.g., chlorine, chlorine dioxide, hydrogen chloride; inorganic fluoride, e.g., hydrogen fluoride; active sulfur compounds, e.g., hydrogen sulfide, elemental sulfur, organic sulfur compounds, sulfur oxides; nitrogen compounds, e.g., nitrogen oxides, ammonia, amines, ammonium ions; ozone; free radicals; organic compounds, e.g., acetic acid, halocarbons, MTBE, carbon dioxide; and/or other undesirable vapors and gases. In certain embodiments, the absorbing agent may emit one or more desirable species. In certain embodiments, the absorbing agent includes a dessicating agent, e.g., a dessicating agent that physically absorbs moisture, is inert and non-water soluble.

Suitable absorbing agents may include, but are not limited to: (1) metals and alloys such as, but not limited to, powdered iron, nickel, copper, aluminum, silicon, solder, silver, gold; (2) metal-plated particulates such as silver-plated copper, silver-plated nickel, silver-plated glass microspheres; (3) inorganics such as BaTiO₃, SrTiO₃, SiO₂, Al₂O₃, ZnO, TiO₂, MnO, CuO, Sb₂O₃ WC, alkali and alkali earth carbonates, fused silica, fumed silica, amorphous fuse silica, sol-gel silica, sol-gel titanates, mixed titanates, ion exchange resins, lithium-containing ceramics, hollow glass microspheres; (4) molecular trap and surface-active materials such as zeolites, silica gels, clays, starches, carbon-based materials such as carbon, activated charcoal, carbon black, ketchem black, diamond powder, fullerenes, derivatized nanotubes; (5) elastomers such as polybutadiene and polysiloxane; and (6) semi-metals and ceramics. In certain instances, the absorbing agent may be calcium oxide. A combination of absorbing agents, i.e., one or more absorbing agents, may be employed, where in certain embodiments two, three or four or more absorbing agents may be employed.

### Channels

As mentioned above, in certain embodiments the absorbing material includes channels such that the channels penetrate through the polymeric base and are open at the surface of the polymeric structures, thus providing access for airborne molecules to the interior portions of the polymer where one or more active components are located. The channeling agents may also provide access for emitted molecules to diffuse out of the polymeric base. These channels may be formed using a variety of chemical and physical or mechanical techniques, or a combination thereof.

Accordingly, in certain embodiments, the subject absorbing materials may also include one or more channeling agents which provides these interconnecting channels or veins that penetrate through the polymeric base. Such channeling agents may not be needed to effectively protect an array from deleterious elements, but may be employed to enhance the capacity of the absorbing material to absorb and/or emit more material over time and thus in certain embodiments may be correctly characterized as capacity-enhancing agents.

Various types of channeling agents may be employed. For example, the channeling agent(s) used in the present invention may generally be any hydrophilic material. In certain embodiments, the hydrophilic material is a polar compound having at least two hydroxy groups. Channeling agents suitable for use in the subject invention include, but are not limited to: hydrophilic, low-melting organic compounds such as polyethylene glycols and polypropylene glycol and mixtures thereof. Other suitable materials include: EVOH, glycerin, pentaerithritol, PVOH, polyvinylpyrollidine, vinylpyrollidone, N-methyl pyrollidone, polysaccharide based compounds such as glucose, fructose (and their alcohols), dextrin, hydrolyzed starch and mannitol are also suitable for the purposes of the present invention.

The capacity of the reactive agent(s) may be enhanced through the use of powdered or sintered materials, or microporous materials, which inherently have channels which can allow gas diffusion to within the polymer material. Accordingly, where appropriate, these channeling agents may be employed in a powdered or sintered form, and not mixed with the base polymer when it is in a molten state. These powdered or sintered materials may be intrinsically absorbing and reactive, such as powdered iron and macromolecular tubes or channels or may be modified to include an agent that absorbs deleterious elements and/or emits beneficial elements.

In certain embodiments, in addition to or in place of using chemical techniques to provide channels, as described above, physical or mechanical techniques may also be employed. For example, the channels may be formed by sintering, drilling, microcracking, laser cutting, and the like, the subject absorbing material to form these channels.

As described above, the subject absorbing materials may be manufactured as inserts to be positioned in appropriate locations within an array holder such that the absorbing material is separately molded and subsequently combined with the array holder or the absorbing material and the device may be a unified piece. That is, the absorbing material may itself be formed into an array holder such that at least a portion of the housing of the holder is made of the absorbing material, i.e., co-molded, and in certain embodiments the entire housing of an array holder is made by forming absorbing material into a housing bottom and/or cover such that the absorbing material forms and functions as a housing. It will be understood that more than one type of absorbing material may be employed in the subject invention, e.g., different absorbing materials may be used, where each is formulated to combat different elements in the environment that are deleterious to an array placed in contact therewith.

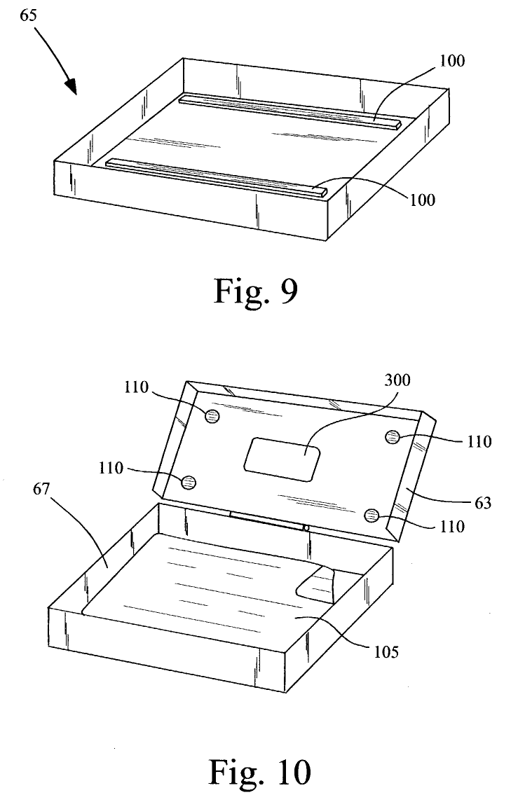

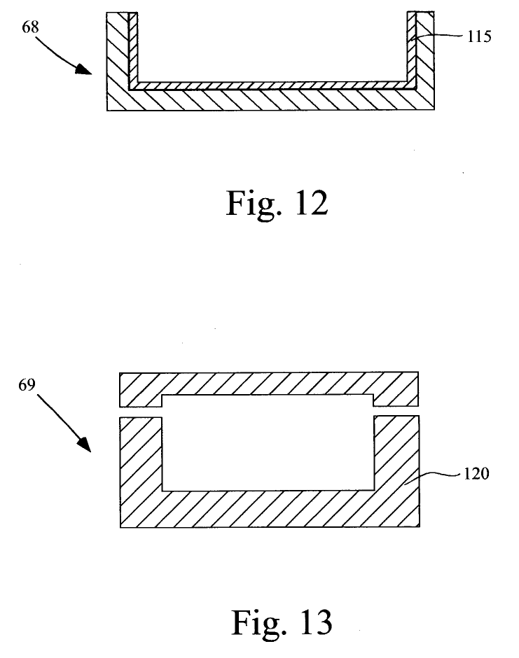

FIGS. 9-13 show exemplary embodiments of the absorbing material in various configurations associated with an array holder housing, where such are not intended to limit the scope of the invention as it will be apparent that the absorbing material may assume various other configurations, as well as be positioned in other locations of the housing than illustrated in these particular FIGS. FIGS. 9, 10 and 11A and 11B show insertable absorbing materials and FIGS. 12 and 13 show absorbing materials that are integral with some or all of the housing.

FIG. 9 shows absorbing material formed as inserts such as one or more insertable strips 100, etc. positioned on a surface of a housing, herein shown positioned on a surface of a housing bottom 65. These insertable materials may be removable and replaceable, as will be described below. FIG. 10 shows absorbing material formed as a sheet or layer 105 (which may be a coating or film) that is positioned on a surface of a housing, herein shown as positioned on a housing bottom 67. As mentioned above, the sheet may be adhered to the housing in a variety of ways. For example, the absorbing material may be shaped and sized to closely approximate that of the area to which it is to be attached and then press fit into place. In addition to or instead of a press fit, the absorbing material may be fixed in place using an adhesive material or shrink-fit protocols. Absorbing material configured as plugs or balls 110 is also associated with the housing cover 63 of FIG. 10, where the absorbing material of the cover may be the same or different from the absorbing material of the bottom.

Also shown in FIG. 10 is an optional indicator 300, for alerting a user when the absorbing material is exhausted and can no longer absorb material. Such an indicator may be a calorimetric indicator or some other display. Accordingly, in certain embodiments an exhausted absorbing material and/or indicator may be removed from the array holder and replaced with another absorbing material and/or indicator, thereby extending the life of an array holder.

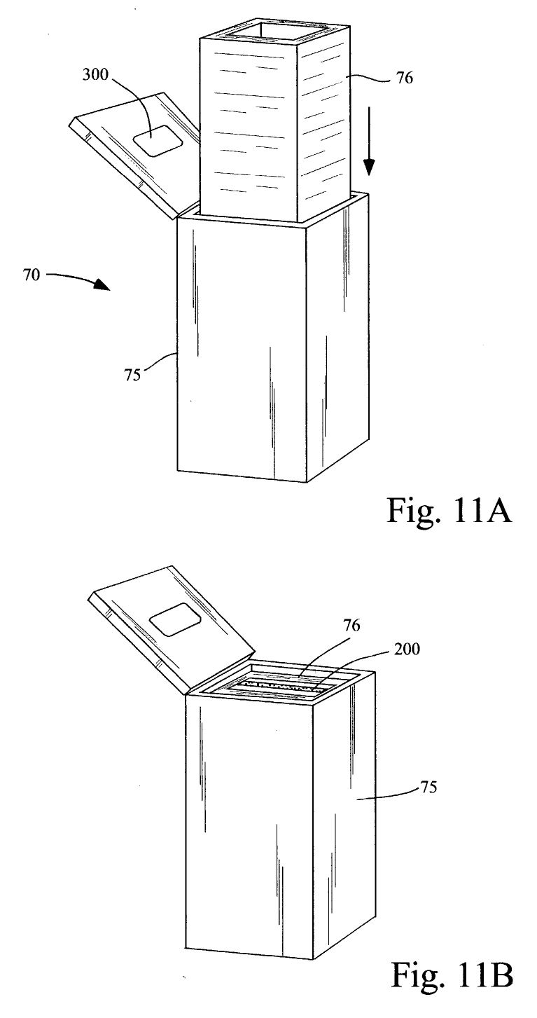

FIGS. 11A and 11B show absorbing material 76 configured as an insertable sleeve or liner, shown in FIG. 11A being inserted into bottom 75 of housing 70 and in FIG. 11B fully inserted into bottom 75 such that array 200 positioned in the bottom is surrounded by the sleeve.

As mentioned above, the absorbing material may be integral with the housing, where such integrated devices may be best viewed in cross section. Accordingly, FIG. 12 shows a cross-sectional view of absorbing agent 115 integrated with the housing, herein shown as integrated with only the bottom 68, such that the bottom and the absorbing material are co-molded. It will be obvious that the entire housing may be co-molded with absorbing material. FIG. 13 shows a cross-sectional view of housing 69 that is made entirely of absorbing material 120 such that the absorbing material is prepared and then formed into a housing, i.e., the absorbing material is configured as the housing.

### Methods of Packaging an Array

Also provided are methods of packaging at least one array in a subject array holder. As described above, the subject array holders are useful throughout the lifecycle of an array, from manufacturing to use of the array at a customer site. Accordingly, an array may be packaged in an array holder at any convenient time and may be repeatedly packaged in a holder during the lifetime of the array such that it is initially packaged in a holder and then removed for a particular purpose and repackaged in the same array holder or in a different subject holder, where this process may be repeated one or more times, e.g., between processing and/or analysis steps.

In general, to package an array a subject holder, as described above, is provided. At least one array is positioned in the interior of the array holder and the holder is sealed around the array(s) positioned inside. In this manner, the absorbing material of the device absorbs molecules deleterious to the array(s) that are in contact with the array(s) inside the sealed device. The seal that is formed may be broken and resealed one or more times to remove and/or add an array, as mentioned above. Usually, the holder provides a hermetic seal. The following method describes an exemplary array lifecycle wherein a subject array holder is used during the entirety of the lifecycle, i.e., an array is associated with a subject array holder throughout each phase of the array's lifecycle, where such is not intended to limit the scope of the invention. It will be apparent that some of the subject steps may be omitted in certain embodiments and/or others may be added. For example, a subject array holder may be employed to hold an array only at certain times during an array's lifecycle and not in others, e.g., only during transport, only at a customer's site, etc.

In certain embodiments, an array may be packaged in an array holder at the point of manufacture of the array, where the array may be held by the array holder (or a different array holder) beginning from the time of manufacture and continuing throughout the lifecycle of the array, i.e., through the useful life of the array. For example, after initially packaging an array in a subject array holder during manufacture, following one or more manufacturing processing steps or manufacturing personnel shift changes or other manufacturing downtimes, if appropriate, the housing may be opened and an array may be removed and subjected to a subsequent manufacturing process. Thereafter, the array may be re-packaged in the array holder to protect it from the harmful atmospheric elements while the array is not being processed. Once manufactured, i.e., after the final manufacturing processing step, the array may be re-packaged in the same or a different holder and transported to a customer site in the array holder to protect the array during this transport phase.

At the customer site, the array, if not immediately used, may be placed, for example, on a shelf or the like in the same array holder in which it was transported or in another subject array holder until it is used. At the time of use, e.g., for use in an array assay or for scanner calibration, the array may be removed from the array holder and used such as in an array assay, calibration procedure, quality control procedure, etc. As mentioned above, after an array assay is performed or if employing an array to calibrate a scanner, the array is scanned by an array scanner to obtain data, e.g., data related to the array assay and/or scanner, etc. However, in many instances the array may not be immediately scanned. In such instances, the array may be re-packaged in the same or different array holder until the time it is scanned so that damage to the array, e.g., to the polymers and/or dye(s) of the array, is minimized.

In certain embodiments an array may be scanned more than one time on separate occasions. The scanning occasions may occur over a period of time, i.e., the times may be remote from each other, where the array may be scanned one or more times in a single day or may be scanned one or more times over a period of days, weeks, months and sometimes years. Accordingly, after an initial scan (and after any subsequent scans), an array may be re-packaged in the same or different array holder to protect it from harmful atmospheric elements between scans. During any point in the array's lifecycle, the active absorbing material and indicator (if used) may be removed from the holder when exhausted and replenished and returned to the holder or replaced with new absorbing material.

In certain embodiments, the array is a scanner calibration array used to calibrate an array scanner or a quality control array to validate and/or verify certain processes. Typically, these arrays are used multiple times over extended periods. In the manner analogous to that described above, these arrays may be packaged and re-packaged one or more times in a subject array holder to protect the arrays between uses.

The subject invention also provides methods for using one or more arrays protected by a subject housing in an array assay such as a hybridization assay or any other analogous binding interaction assay. Generally in these binding assays, a sample suspected of including an analyte of interest, i.e., a target molecule, is contacted with an array under conditions sufficient for the analyte target in the sample to bind to its respective binding pair member that is present on the array. Thus, if the analyte of interest is present in the sample, it binds to the array at the site of its complementary binding member and a complex is formed on the array surface. The presence of this binding complex on the array surface is then detected, e.g., through use of a signal production system, e.g., an isotopic or fluorescent label or the like present on the analyte, as described above. The presence of the analyte in the sample is then deduced from the detection of binding complexes on the substrate surface.

As mentioned above, arrays held in a subject array holder may be used in a variety of array-based assays, where hybridization reactions will be used herein for exemplary purposes only, and is not intended to limit the scope of the invention. In hybridization assays, a sample of target analyte such as target nucleic acids is first prepared, where preparation may include labeling of the target nucleic acids with a label, e.g., with a member of signal producing system, and the sample is then contacted with the array, e.g., a nucleic acid array, under hybridization conditions, whereby complexes are formed between target analytes such as nucleic acids that are complementary to probe sequences attached to the array surface. The presence of hybridized complexes is then detected as described above. Specific hybridization assays of interest which may be practiced using the subject methods include: gene discovery assays, differential gene expression analysis assays; nucleic acid sequencing assays, and the like. Patent applications describing methods of using arrays in various applications include: WO 95/21265; WO 96/31622; WO 97/10365; WO 97/27317; EP 373 203; and EP 785 280 and U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of the U.S. patents which are herein incorporated by reference.

Accordingly, in practicing the subject methods the first step is to provide a subject array holding device having at least one array sealed therein, as described above, such that the absorbent material associated with the array holder absorbs molecules deleterious to the array(s) sealed in the interior of the device. The held array(s) may be positioned and sealed in the array holder at any time prior to the time the array is used in an array assay, as described above, e.g., at the manufacturing site, during transport, at the customer site, etc., where the seal is typically a hermetic seal.

Accordingly, the subject methods may also include packaging at least one array in a subject array holding device at a first site, e.g., a manufacturing facility or the like, and transporting the packaged array to a second remote site for use in an array assay. For example, the array may be packaged in an array holder at a remote manufacturing site and transported to a second customer site. By “second site” in this context is meant a site other than the site at which the array is packaged in the array holder. For example, a second site could be another site (e.g., another office, lab, etc.) in the same building, city, another location in a different city, another location in a different state, another location in a different country, etc. Usually, though not always, the first site and the second site are at least in different buildings, and may be at least one mile, ten miles, or at least one hundred miles apart.

Once the one or more arrays are operatively sealed in the array holder such that they are protected from harmful atmospheric elements, the seal of the device is broken and one or more arrays are removed from the device for use. At this point, if one or more arrays remain in the holder, the holder may, in certain embodiments, be re-sealed, e.g., re-sealed to provide a hermetic seal, so that the arrays therein may continue to be protected form harmful elements. The one or more arrays that have been removed from the device are contacted with a fluid sample suspected of containing target analyte, e.g., target nucleic acids, that are complementary to probe sequences attached to the array surface. As will be apparent to those of skill in the art, the sample may be any suitable sample which is suspected of including a member of a specific binding pair. That is, the sample may be a sample that is suspected of containing molecules capable of binding with a biopolymeric probe bound to the surface of the substrate. The sample may include the target analyte, often pre-amplified and labeled.

Thus, at some time prior to the detection step, described below, any target analyte present in the initial sample contacted with the array may be labeled with a detectable label. Labeling can occur either prior to or following contact with the array. In other words, the analyte, e.g., nucleic acids, present in the fluid sample contacted with the array may be labeled prior to or after contact, e.g., hybridization, with the array. In some embodiments of the subject methods, the sample analytes e.g., nucleic acids, are directly labeled with a detectable label, wherein the label may be covalently or non-covalently attached to the nucleic acids of the sample. For example, the nucleic acids, including the target nucleotide sequence, may be labeled with biotin, exposed to hybridization conditions, wherein the labeled target nucleotide sequence binds to an avidin-label or an avidin-generating species. In an alternative embodiment, the target analyte such as the target nucleotide sequence is indirectly labeled with a detectable label, wherein the label may be covalently or non-covalently attached to the target nucleotide sequence. For example, the label may be non-covalently attached to a linker group, which in turn is (i) covalently attached to the target nucleotide sequence, or (ii) comprises a sequence which is complementary to the target nucleotide sequence. In another example, the probes may be extended, after hybridization, using chain-extension technology or sandwich-assay technology to generate a detectable signal (see, e.g., U.S. Pat. No. 5,200,314). Generally, such detectable labels include, but are not limited to, radioactive isotopes, fluorescers, chemiluminescers, enzymes, enzyme substrates, enzyme cofactors, enzyme inhibitors, dyes, metal ions, metal sols, ligands (e.g., biotin or haptens) and the like.

In one embodiment, the label is a fluorescent compound, i.e., capable of emitting radiation (visible or invisible) upon stimulation by radiation of a wavelength different from that of the emitted radiation, or through other manners of excitation, e.g. chemical or non-radiative energy transfer. The label may be a fluorescent dye. Usually, a target with a fluorescent label includes a fluorescent group covalently attached to a nucleic acid molecule capable of binding specifically to the complementary probe nucleotide sequence.

The sample may be introduced to the one or more arrays using any convenient protocol, e.g., sample may be introduced using a pipette, syringe or any other suitable introduction protocol. The sample is contacted with the array under appropriate conditions to form binding complexes on the surface of the substrate by the interaction of the surface-bound probe molecule and the complementary target molecule in the sample. In the case of hybridization assays, the sample is typically contacted with an array under stringent hybridization conditions, whereby complexes are formed between target nucleic acids that are complementary to probe sequences attached to the array surface, i.e., duplex nucleic acids are formed on the surface of the substrate by the interaction of the probe nucleic acid and its complement target nucleic acid present in the sample. An example of stringent hybridization conditions is hybridization at about 50° C. or higher and 0.1×SSC (15 mM sodium chloride/1.5 mM sodium citrate). Another example of stringent hybridization conditions is overnight incubation at about 42° C. in a solution: 50% formamide, 5×SSC (150 mM NaCl, 15 mM trisodium citrate), 50 mM sodium phosphate (pH7.6), 5×Denhardt's solution, 10% dextran sulfate, followed by washing the arrays in 0.1×SSC at about 65° C. Hybridization involving nucleic acids generally takes from about 30 minutes to about 24 hours, but may vary as required. Stringent hybridization conditions are hybridization conditions that are at least as stringent as the above representative conditions, where conditions are considered to be at least as stringent if they are at least about 80% as stringent, typically at least about 90% as stringent as the above specific stringent conditions. Other stringent hybridization conditions are known in the art and may also be employed as appropriate, as well as other less stringent conditions.

Once the incubation step is complete, the array is washed at least one time to remove any unbound and non-specifically bound sample from the substrate, generally at least two wash cycles are used. Washing agents used in array assays are known in the art and, of course, may vary depending on the particular binding pair used in the particular assay. For example, in those embodiments employing nucleic acid hybridization, washing agents of interest include, but are not limited to, salt solutions such as sodium, sodium phosphate and sodium, sodium chloride and the like as is known in the art, at different concentrations and may include some surfactant as well.

Following the washing procedure, the at least one array is then interrogated or read so that the presence of the binding complexes is then detected i.e., the label is detected using calorimetric, fluorimetric, chemiluminescent or bioluminescent methods. In certain embodiments, there may be a delay between the time the array assay is completed and the reading of the array. In such cases, an array awaiting scanning may be stored in a subject array holder to protect it until the time it is to be scanned, where at that time the holder may be removed and the array subjected to scanning.

Reading of the at least one array may be accomplished by illuminating the at least one array and reading the location and intensity of resulting fluorescence at each feature of the array to obtain a result. For example, a scanner may be used for this purpose, which is similar to the MICROARRAY SCANNER available from Agilent Technologies, Palo Alto, Calif. Other suitable apparatus and methods for reading an array are described in U.S. patent application Ser. Nos.: Ser. No. 20/087447 “Reading Dry Chemical Arrays Through The Substrate” by Dorsal et al., Ser. No. 09/846125 “Reading Multi-Featured Arrays” by Dorsel et al.; and Ser. No. 09/430214 “Interrogating Multi-Featured Arrays” by Dorsel et al., the disclosures of which are herein incorporated by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. Nos. 6,251,685; 6,221,583, the disclosure of which is herein incorporated by reference, and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or whether or not a pattern indicates a particular condition of an organism from which the sample came). The results of the reading (whether further processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” it is meant a location other than the location at which the array is present and the array assay, e.g., hybridization, occurs. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, Internet, etc.

### Kits

Finally, kits which include the subject array holding devices are provided. The subject kits at least include one or more subject array holding devices. In many instances the kits will also include one or more arrays such as nucleic acid or peptide arrays. In many embodiments the array holders will have at least one array packaged therein. The kits may further include one or more additional components necessary for carrying out an analyte detection assay, such as sample preparation reagents, buffers, labels, and the like. As such, the kits may include one or more containers such as vials or bottles, with each container containing a separate component for the assay, and reagents for carrying out an array assay such as a nucleic acid hybridization assay or the like. The kit may also include a denaturation reagent for denaturing the analyte, buffers such as hybridization buffers, wash mediums, enzyme substrates, reagents for generating a labeled target sample such as a labeled target nucleic acid sample, negative and positive controls and written instructions for using the subject array assay devices for carrying out an array based assay. The instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., CD-ROM, diskette, etc.

### Experimental

The following example is put forth so as to provide those of ordinary skill in the art with a complete disclosure and description of how to make and use the present invention, and are not intended to limit the scope of what the inventors regard as their invention. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Experiments were performed using arrays packaged in a housing having an absorbing agent in communication with the packaged array (the “test array”). A first experiment was performed under room temperature conditions and a second, accelerated aging experiment was performed under elevated temperatures. One or more control packages were also employed in each experiment that did not include any absorbing agent (the “control array”). Over a period of about 3-5 months (four weeks for the accelerated aging experiment), both the test arrays and the control arrays were removed from the housing and an area having certain features of each array was scanned to determine the intensity of the features over time. The results of the experiments, described in greater detail below, showed that the absorbing material protected the test arrays, and more specifically the oligonucleotide probes as well as the dye label, from degradation such that no significant loss of signal was observed over the time period of the tests. However, the control arrays showed loss of signal over the time period of the tests.

Array assays for each experiment were performed according to the following protocol:

Samples were fragmented with a solution of zinc acetate in TRIS (ph 7.6).

The arrays were contacted with the sample of targets and incubated at about 44° C. for about 17 hours in a solution of Sodium-MES (pH 6.4). The arrays were then removed and washed in room temperature 6×SSPE/0.005% Sarcosine and 0.06×SSPE solutions.

### A. Room Temperature Experiment

### I. Room Temperature Protocol

Standard arrays of oligonucleotides probes were provided by Agilent Technologies, Inc. of Palo Alto, Calif. An array assay was performed by exposing the arrays to Cy3- and Cy5-labeled target molecules that were complementary and which bound to the probe features of the arrays when contacted thereto. The arrays were separated into three groups: (1) 1238 test arrays, (2) 1239 test arrays, and (3) control arrays. Absorbing material provided by CSP Technologies of Alabama (part nos. 1238 and 1239) was cut into strips having dimensions of about 1.5″ by about 3″ and placed into the respective packages such that the 1238 absorbing material was placed in the 1238 array package and the 1239 absorbing material was placed in the 1239 array package. No absorbing material was placed in the control package. The packages were made of flexible, metalized polyethylene and had resealable closures. All of the arrays were scanned for a baseline reading immediately after being contacted with target molecules. Following the baseline scan, the arrays, whether test or control, were sealed in their respective array packages—one package for each array. The packages were maintained at room temperature throughout the test period.

Over about the next three months (five months for the 1239 arrays), the packages were unsealed and the arrays were removed, scanned and then re-sealed in the same package with the same absorbing material. The time points were chosen to be about 30 days apart from one another for the three month studies and about five months apart for the five month studies. To minimize exposure to the air, the arrays were removed one at a time from their packages, scanned and immediately re-packaged. The whole area of each array was scanned each month, and a small area on each array was analyzed. The analyzed area included a certain number of features, typically up to about 100 features, representative of all the features on the array. All scans were performed on the same scanner (Agilent Technologies G2505A MICROARRAY SCANNER from Agilent Technologies, Inc. of Palo Alto, Calif.).

The arrays were analyzed by Agilent Technologies' commercially available feature extraction software program, version 4.1.1 and which is compatible with the scanner employed. Generally, this analysis observes the fluorescence intensities of each feature, compares it to background signal and provides a net signal for each feature.

### II. Room Temperature Results

The test arrays showed no significant loss of signal over the testing period. The signal levels of the test arrays between features are constant to within the scanner's stability level (approximately 1-2% is expected). Each test feature, regardless of chemical composition, showed about a 1-2% variation from scan to scan. Such variation is within the typical variation inherent in the test and is likely a result of the algorithm employed to analyze the arrays.

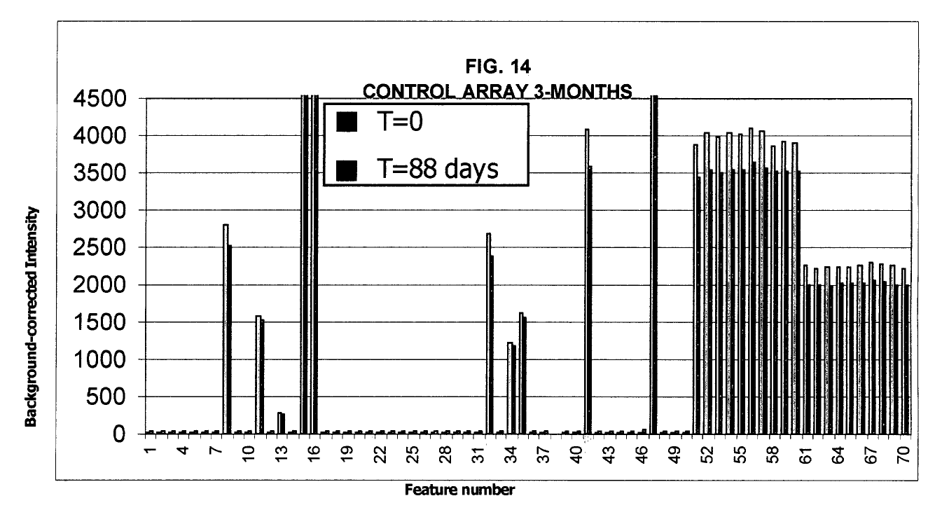

FIGS. 14, 15A, 15B, 16A and 16B illustrate the results of the experiment presented in chart format where fluorescent intensity is represented on the y-axis and feature number is represented on the x-axis for each array. The number of columns per each feature number represents the various scanning events over about a three month period. In order to provide optimum resolution of the charts, not all of the features that were scanned are shown. Certain feature numbers are shown terminating at the highest signal intensity for ease of illustration only, where the signal levels of these features were actually higher than shown.

As mentioned above, sometimes loss of signal is observed when arrays are exposed to deleterious elements in the environment, shown for example in FIG. 14 which illustrates the results of a control array over about three months (two time points). As shown, degradation of the signal occurred over this time period.

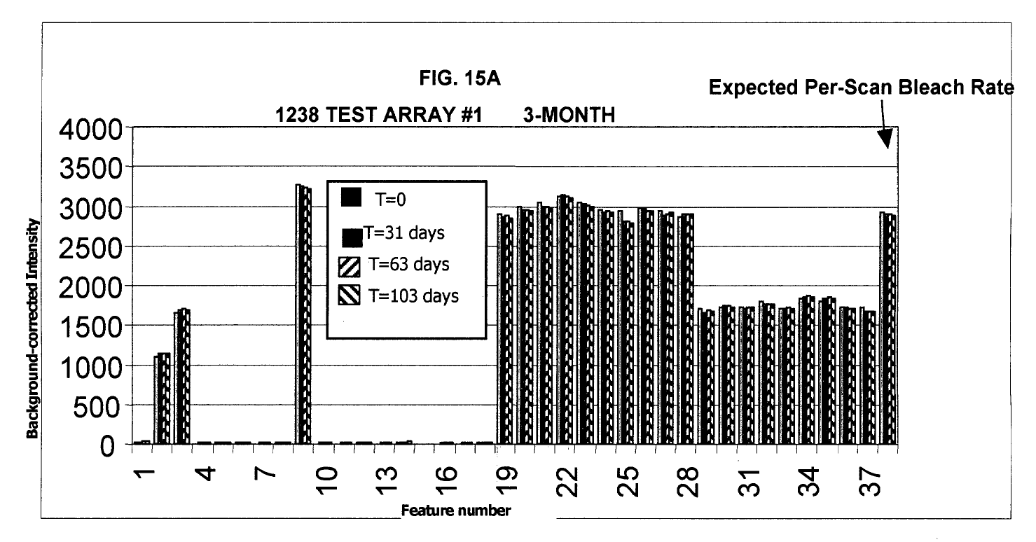

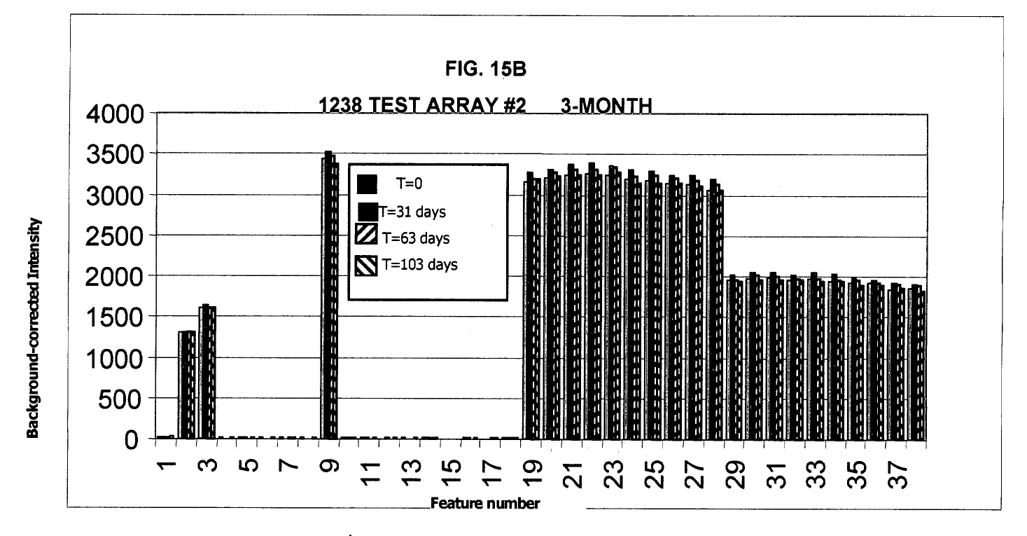

FIGS. 15A and 15B show two different 1238 test arrays, respectively, over about three months (four time points). As shown, no significant degradation of signal is observed for any of the features, i.e., the dye has been protected by the absorbing material. Also shown in FIG. 15A, for comparison, is the expected loss in signal that is expected from photobleaching by the scanner.

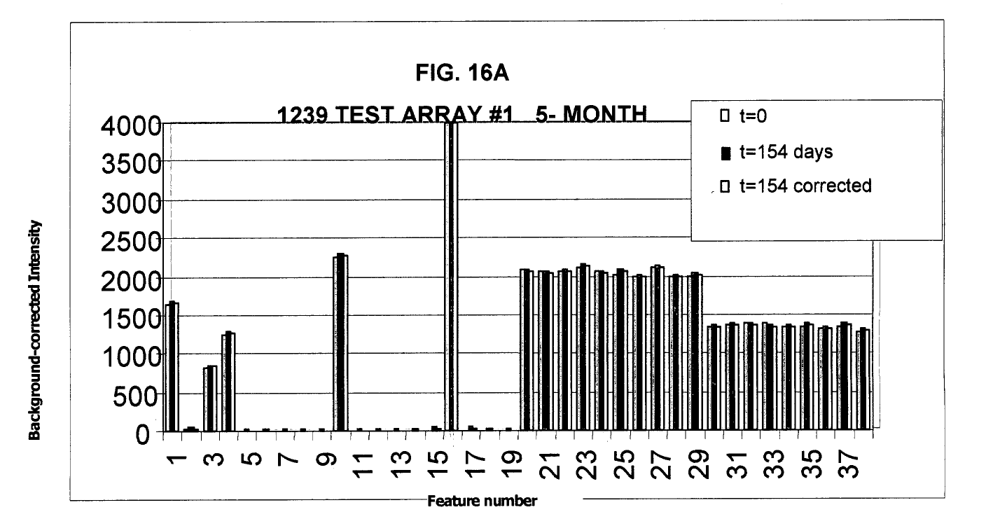

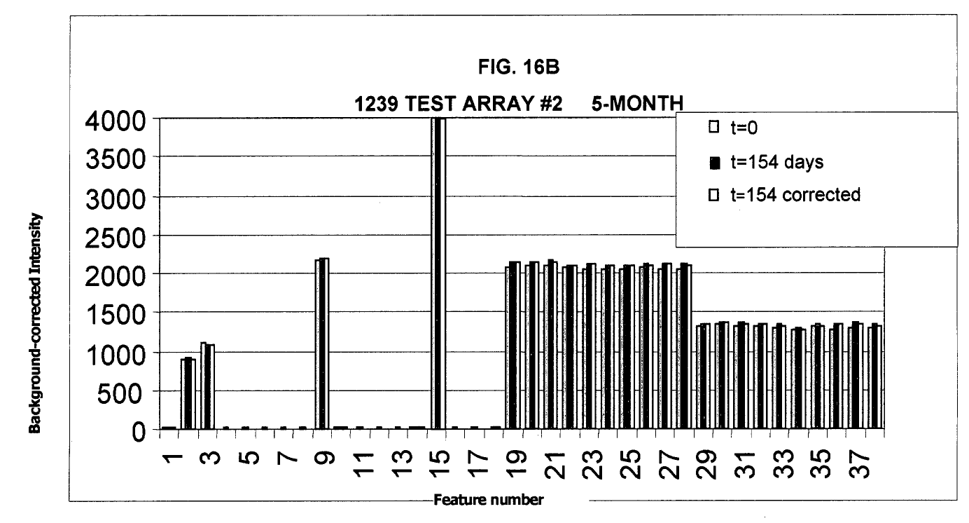

FIGS. 16A and 16B show two different 1239 test arrays, respectively, over about five months (two time points). As shown, no significant degradation of signal is observed for any of the features, i.e., the dye has been protected by the absorbing material. A correction factor was subtracted from the intensities of all features from the second time point due to a molecule emitted from the package. This correction did not alter the overall results of the test as the correction lowered, i.e., was subtracted from, the signal intensity. Thus, even with the correction the signals did not show significant loss over the test period.

### B. Accelerated Aging Experiment

### I. Accelerated Aging Protocol

An accelerated aging experiment was performed over about a four week period at about 55° C. This test was designed to determine whether arrays not exposed to binding target molecules would show degradation if stored in a protected environment according to the subject invention. Typically, when simply stored in a vacuum or nitrogen at 55° C. for four weeks (i.e., not packaged and stored according to the subject invention), the array features lose much of their ability to combine with their target molecules.

Arrays having no protective material included were employed as controls. In this test, a first set of arrays (“A” test arrays ) were packaged according to the subject invention, i.e., packaged with absorbing agent, for one week at about 55° C. prior to be used in an array assay, i.e., prior to being contacted with labeled target. A second set of arrays (“B” test arrays), already exposed to Cy3/Cy5-labeled target molecules, were packaged according to the subject invention, i.e., packaged with absorbing agent, for four weeks at about 55° C., and tested twice during that time. A third set of arrays (“C” test arrays), were packaged according to the subject invention, i.e., packaged with absorbing agent, for four weeks at about 55° C., prior to being used in an array assay, i.e., prior to being contacted with labeled target. Two sets of arrays were used as controls; one control set was packaged in the same type of housing package as arrays A, B and C, but without absorbing agent and with vacuum and the other control was packaged in the same type of housing package as arrays A, B and C, but without absorbing agent and with nitrogen. Each control group included (1) arrays that were immediately used in an array assay and scanned, (2) arrays that were packaged and stored prior to being used in an array assay, removed from the package and used in an array assay and scanned, and (3) arrays that were packaged and stored prior to being used in an array assay, removed from the package and used in an array assay and scanned, and then repackaged, stored, removed again and rescanned.

After about one week, the “A” and “B” arrays were removed from the storage environment. “A” arrays were removed from their package and used in an array assay, i.e., hybridized to target molecules. Following this hybridization assay, the “A” arrays were scanned. The “B” arrays were removed from their packaging one at a time, also scanned, and immediately re-packaged and stored until the next time point. The “C” arrays, were processed in the same way as the “A” arrays, after four weeks had passed. As such, at four weeks, the “C” arrays were scanned, as well as the “B” arrays re-scanned.

### II. Accelerated Aging Results

The results showed that after about four weeks, there was no significant degradation of signal observed for any of the “A”, “B” and “C” arrays. That is, the oligonucleotide probes as well as the dye molecules have been protected by the absorbing material. However, all of the vacuum control arrays and the nitrogen control arrays showed significant loss of signal over the time period of the test.

It is evident from the above results and discussion that the above described invention provides devices and methods for holding and storing at least one array in a protective package such that the arrays are not in constant contact with elements in the atmosphere that are harmful to the arrays. The above-described invention provides a number of advantages including cost effectiveness, ease of use, effectiveness at providing a controlled atmosphere for the arrays and the ability to be employed in various phases of an array's lifetime. As such, the subject invention represents a significant contribution to the art.

All publications and patents cited in this specification are herein incorporated by reference as if each individual publication or patent were specifically and individually indicated to be incorporated by reference. The citation of any publication is for its disclosure prior to the filing date and should not be construed as an admission that the present invention is not entitled to antedate such publication by virtue of prior invention.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A device for holding at least one array therein, said device comprising:
(a) a housing having at least one array therein; and
(b) an absorbing material associated with said housing, whereby said absorbing material is capable of absorbing molecules within said housing deleterious to said at least one array.

2. The device according to claim 1, wherein said absorbing material is comprised of a polymer and an absorbing agent.

3. The device according to claim 1, wherein said polymer is chosen from the group of:
polypropylenes, polyethylenes, polyisoprenes, polybutadienes, polybutenes, polysiloxanes, polycarbonates, polyamides, ethylene-vinyl acetate copolymers, ethylene-methacrylate copolymers, poly(vinyl chloride), polystyrenes, polyesters, polyanhydrides, polyacrylonitriles, polysulfones, polyacrylic esters, acrylics, polyurethanes and polyacetals, polypropylene maleic anhydrides, polyethylene maleic anhydrides, polystyrene maleic anhydrides, polyethylene acrylic acids, polyethylene-uretheres, polyethylene-EVOH, polyethylene-nylons, grafted polyolefins, polyamides, ethylene-vinyl acetate partially hydrolyzed polymers, ethylene-methacrylate partially hydrolyzed polymers, grafted polyvinyl chlorides, grafted polystyrenes, polyesters, polyester amides, polyacrylic partially hydrolyzed esters, acrylics, polyurethanes, polyacetals, adhesives.

4. The device according to claim 1, wherein said absorbing agent is capable of absorbing at least one of: water, acids, bases, corrosives, oxidizing gases, free radicals and reducing gases.

5. The device according to claim 1, wherein said absorbing agent is capable of absorbing at least one of: inorganic chlorides, inorganic fluorides, sulfur compounds, nitrogen compounds, ozone, and organic compounds.

6. The device according to claim 2, wherein said absorbing agent is chosen from the group of metals, metal alloys, metal-plated particulates, inorganic molecules, molecular traps, surface-active materials, elastomers, semi-metals and ceramics.

7. The device according to claim 2, wherein said absorbing agent is chosen from the group of powdered iron, nickel, copper, aluminum, silicon, solder, silver, gold, silver-plated copper, silver-plated nickel, silver-plated glass microspheres, BaTiO₃, SrTiO₃, SiO₂, Al₂O₃, ZnO, TiO₂, MnO, CuO, Sb₂O₃ WC, alkali carbonates, alkali earth carbonates, fused silica, fumed silica, amorphous fuse silica, sol-gel silica, sol-gel titanates, mixed titanates, ion exchange resins, lithium-containing ceramics, hollow glass microspheres, zeolites, silica gels, clays, starches, carbon-based materials, carbon, activated charcoal, carbon black, ketchem black, diamond powder, fullerenes, derivatized nanotubes, polybutadienes, polysiloxanes, and calcium oxides.

8. The device according to claim 1, wherein said device comprises channels.

9. The device according to claim 8, wherein said channels are formed by at least one of a channeling agent, sintering, drilling, microcracking and laser cutting.

10. The device according to claim 9, wherein said channeling agent is hydrophilic.

11. The device according to claim 9, wherein said channeling agent is chosen from the group of polyethylene glycols, polypropylene glycols, mixtures of polyethylene glycols and polypropylene glycols, EVOH, glycerin, pentaerithritols, PVOH, polyvinylpyrollidines, vinylpyrollidone, N-methyl pyrollidones, and polysaccharides.

12. The device according to claim 1, wherein said housing is capable of forming a hermetic seal.

13. The device according to claim 12, wherein said seal is resealable.

14. The device according to claim 1, further comprising an emitting material associated with said housing that is capable of emitting molecules advantageous to said at least one array.

15. The device according to claim 14, wherein said emitting material is chosen from the group of antioxidants, anti-reducing agents, anti-acid agents and anti-radical agents.

16. The device according to claim 1, wherein said at least one array is an array of polymers.

17. The device according to claim 16, wherein said polymers are nucleic acids.

18. The device according to claim 16, wherein said polymers are peptides.

19. The device according to claim 1, wherein said absorbing material is in the form of a sheet, strip, film, layer, coating, laminate or plug.

20. The device according to claim 1, wherein said absorbing material is integral to said housing.

21. The device according to claim 20, wherein said absorbing material is co-molded with said housing.

22. The device according to Clam 1, wherein said absorbing material forms said housing.

23. The device according to claim 1, wherein said absorbing material is an insertable sleeve.

24. A method of packaging at least one array, said method comprising:
(a) providing an array holding device, said array holding device comprising:
(i) a housing having at least one array therein, and
(ii) an absorbing material associated with said housing, whereby said absorbing material is capable of absorbing molecules within said housing deleterious to said at least one array;
(b) positioning at least one array inside said array holding device; and
(c) sealing said at least one array inside said array holder, whereby said absorbing material absorbs molecules deleterious to said at least one array.

25. The method according to claim 24, wherein said seal is a hermetic seal.

26. The method according to claim 24, wherein said housing further comprises an emitting agent capable of emitting molecules within said housing that are beneficial to said at least one array.

27. The method according to claim 24, further comprising breaking and resealing said seal.

28. A method of performing an array assay, said method comprising:
(a) providing an array holding device according to claim 1;
(b) sealing said at least one array in said array holding device, whereby said absorbing materials absorbs molecules within said housing deleterious to said at least one array;
(c) breaking said seal and removing said at least one array from said device; and
(d) contacting said removed at least one array with sample to perform an array assay with said removed at least one array.

29. The method according to claims 28, further comprising reading said at least one array to obtain a result.

30. The method according to claim 29, further comprising forwarding data representing a result of said reading from a first location to a second location.

31. The method according to claim 30, wherein said second location is remote from said first location.

32. The method according to claim 29, further comprising receiving data representing a result of said reading.

33. The method according to claim 28, further comprising, after performing said array assay, resealing said at least one array in the same or different array holder and at some time thereafter breaking said seal and removing said resealed at least one array from said device.

34. The method according to claim 28, wherein said array holding device comprises more than one array and said method further comprises repeating steps B through C one or more times for any remaining arrays therein.

35. A kit for performing an array assay, said kit comprising:
(a) at least one device according to claim 1,
(b) instructions for using said at least one array in an array assay.
